(19) United States
(12) Patent Application Publication
(10) Pub. No.: US 2021/0353570 A1
(43) Pub. Date: Nov. 18, 2021

(54) Flutamide Microsphere-Based Arterial Embolization for Treating Prostate Disorders

(71) Applicant: Biovena Science, LLC, New York, NY (US)

(72) Inventors: Fuad Nurili, New York, NY (US); Omer Aras, Edgewater, NJ (US)

(21) Appl. No.: 17/277,400

(22) PCT Filed: Sep. 19, 2019
(86) PCT No.: PCT/US2019/051898

§ 371 Date: Mar. 18, 2021

Related U.S. Application Data
(60) Provisional application No. 62/733,710, filed on Sep. 20, 2018.

(51) Int. Cl. A61K 31/167; A61K 9/16; A61P 13/08; A61B 17/12

(52) U.S. Cl. CPC A61K 31/167; A61K 9/1647; A61B 17/12186; A61B 17/1204; A61B 17/12109; A61P 13/08

(57) ABSTRACT

This invention provides methods for treating prostate cancer and benign prostatic hyperplasia. Each method comprises introducing biodegradable microspheres into one or more of a subject's prostate arteries, wherein the microspheres (i) have a d90 value from 40 gm to 500 gm; (ii) comprise polylactic acid (PLA) and/or polylactic co-glycolic acid (PLGA); (iii) carry a therapeutically effective amount of pharmaceutical flutamide; (iv) embolize prostate arterial vessels supplied by the one or more arteries into which they are introduced; and (v) release flutamide during embolization. This invention also provides flutamide-carrying biodegradable microspheres and related articles of manufacture.

This application claims the benefit of U.S. Provisional Application No. 62/733,710, filed Sep. 20, 2018, the contents of which are incorporated herein by reference.

Throughout this application, various publications are cited. The disclosure of these publications is hereby incorporated by reference into this application to describe more fully the state of the art to which this invention pertains.

### FIELD OF THE INVENTION

The present invention relates to methods for treating prostate disorders via prostate arterial embolization. These methods employ flutamide-carrying biodegradable microspheres.

### BACKGROUND OF HE INVENTION

Prostate Cancer and Benign Prostatic Hyperplasia

Benign Prostatic Hyperplasia (“BPH”) is one of the most common diseases of older men, with more than 70% of men over 70 years of age affected worldwide. In 2010, BPH affected 210 million men worldwide, or about six percent of the male population, according to a 2012 report in the Lancet. Prostate cancer is the most common cancer in men in the world. The exact etiology of both these prostate diseases has yet to be fully elucidated. Both diseases have striking similarities regarding androgen-dependence driving their pathophysiology, and inflammatory components that contribute to the development of the diseases.

Flutamide

Flutamide is an anti-androgenic drug. It is indicated as a monotherapy for the treatment of patients with locally advanced or metastatic prostate cancers and BPH. It is a potent inhibitor of testosterone-stimulated prostatic DNA synthesis, and it inhibits the growth of prostate cancer cells. Flutamide is a Biopharmaceutical Classification System class II drug, and it undergoes extensive first-pass effect through CYP1A2 after oral administration. It has a short half-life of 5-6 hours (log P 3.350), with low solubility and bioavailability (49%).

Microspheres

Biodegradable microspheres made of polylactic co-glycolic acid (PLGA) and polylactic acid (PLA) are known and suitable for transarterial drug delivery and transient embolization. PLGA is an FDA-approved biodegradable polymer. It has been extensively investigated in many medical and pharmaceutical fields due to its good biodegradability and biocompatibility. PLGA-containing microspheres have shown sustained release characteristics due to degradation and diffusion mechanisms. Biodegradability advantages include potential reduction in the occurrence of post-embolization syndrome tissue inflammation and fibrosis, risks arising from non-target embolization, and the possibility of repeated interventions after vessel recanalization.

Embolization

Prostate arterial embolization (PAE) is a procedure developed over the last decade. Numerous papers on it have been published. By way of example, Pisco, et al. (2016) discusses prostate embolization as an alternative to open surgery in patients with large prostate and moderate to severe lower urinary tract symptoms. Gao, et al. (2013) discusses prostatic arterial embolization for BPH versus transurethral resection of the prostate in the context of a prospective, randomized, and controlled clinical trial. And, Young, et al. (2017) teaches that prostate arterial embolization is a viable option for treating symptoms of BPH.

An Unmet Need

There is an unmet need for a superior way to employ prostate embolization to treat prostate cancer and BPH.

### SUMMARY OF THE INVENTION

This invention provides a method for treating a prostate disorder in a subject comprising introducing biodegradable microspheres into one or more of the subject's prostate arteries, wherein the microspheres (i) have a d₉₀ value from 40 μm to 500 μm; (ii) comprise polylactic acid (PLA) and/or polylactic co-glycolic acid (PLGA); (iii) carry a therapeutically effective amount of pharmaceutical flutamide; (iv) embolize prostate arterial vessels supplied by the one or more arteries into which they are introduced; and (v) release flutamide during embolization.

This invention also provides a method for treating prostate cancer in a human subject comprising introducing biodegradable microspheres into the subject's prostatic artery, wherein the microspheres (i) have a d₉₀ value from 70 μm to 150 μm; (ii) comprise FLA and PLGA at a PLA:PLGA molar ratio of 45:55; (iii) carry from 500 mg to 2,000 mg of pharmaceutical flutamide; (iv) embolize prostate arterial vessels supplied by the prostatic artery; and (v) release flutamide during embolization.

This invention further provides a method for treating benign prostatic hyperplasia in a human subject comprising introducing biodegradable microspheres into the subject's prostatic artery, wherein the microspheres (i) have a d₉₀ value from 70 μm to 150 μm; (ii) comprise PLA and PLGA at a PLA:PLGA molar ratio of 45:55; (iii) carry from 500 mg to 2,000 mg of pharmaceutical flutamide; (iv) embolize prostate arterial vessels supplied by the prostatic artery; and (v) release flutamide during embolization.

This invention provides a biodegradable microsphere, wherein the microsphere (I) has a diameter of from 40 μm to 500 μm; (ii) comprises polylactic acid (FLA) and/or polylactic co-glycolic acid (PLGA); (iii) carries pharmaceutical flutamide; (iv) embolizes a prostate arterial vessel when introduced into an artery that supplies it; and (v) releases flutamide during embolization.

This invention also provides a plurality of biodegradable microspheres, wherein the microspheres (i) have a d₉₀ value from 40 μm to 500 μm; (ii) comprise polylactic acid (PLA) and/or polylactic co-glycolic acid (PLGA); (iii) carry a therapeutically effective amount of pharmaceutical flutamide; (iv) embolize prostate arterial vessels when introduced into one or more arteries that supply them; and (v) release flutamide during embolization.

This invention further provides an article of manufacture for use in treating prostate cancer by introducing biodegradable microspheres into one or more of the subject's prostate arteries. The article comprises (a) biodegradable microspheres, wherein the microspheres (i) have a d₉₀ value from 40 μm to 500 μm, (ii) comprise polylactic acid (PLA) and/or polylactic co-glycolic acid (PLGA), (iii) carry a therapeutically effective amount of pharmaceutical flutamide, (iv) embolize prostate arterial vessels supplied by the one or more prostate arteries into which they are introduced, and (v) release flutamide during embolization; and (b) a label instructing the user to introduce the biodegradable microspheres into one or more of a human subject's prostate arteries so as to treat prostate cancer in the subject.

This invention still further provides an article of manufacture for use in treating prostate cancer in a human subject by introducing biodegradable microspheres into the subject's prostatic artery. The article comprises (a) biodegradable microspheres, wherein the microspheres (i) have a d₉₀ value from 70 μm to 150 μm, (ii) comprise PLA and PLGA at a PLA:PLGA molar ratio of 45:55, (iii) carry from 500 mg to 2,000 mg of pharmaceutical flutamide, (iv) embolize prostate arterial vessels supplied by the prostatic artery, and (v) release flutamide during embolization; and (b) a label instructing the user to introduce the biodegradable microspheres into a human subject's prostatic artery so as to treat prostate cancer in the subject.

This invention also provides an article of manufacture for use in treating benign prostatic hyperplasia by introducing biodegradable microspheres into one or more of the subject's prostate arteries. The article comprises (a) biodegradable microspheres, wherein the microspheres (i) have a d₉₀ value from 40 μm to 500 μm, (ii) comprise polylactic acid (PLA) and/or polylactic co-glycolic acid (PLGA), (iii) carry a therapeutically effective amount of pharmaceutical flutamide, (iv) embolize prostate arterial vessels supplied by the one or more prostate arteries into which they are introduced, and (v) release flutamide during embolization; and (b) a label instructing the user to introduce the biodegradable microspheres into one or more of a human subject's prostate arteries so as to treat benign prostatic hyperplasia in the subject.

Finally, this invention provides an article of manufacture for use in treating benign prostatic hyperplasia in a human subject by introducing biodegradable microspheres into the subject's prostatic artery. The article comprises (a) biodegradable microspheres, wherein the microspheres (i) have a d₉₀ value from 70 μm to 150 μm, (ii) comprise PLA and PLGA at a PLA:PLGA molar ratio of 45:55, (iii) carry from 500 mg to 2,000 mg of pharmaceutical flutamide, (iv) embolize prostate arterial vessels supplied by the prostatic artery, and (v) release flutamide during embolization; and (b) a label instructing the user to introduce the biodegradable microspheres into a human subject's prostatic artery so as to treat benign prostatic hyperplasia in the subject.

### BRIEF DESCRIPTION OF THE FIGURES

FIG. 1

The left side of this figure shows an illustration of the catheter-directed delivery of flutamide-loaded PLA/PLGA particles to prostatic tissue via the transarterial route. The right side of this figure shows a digital subtraction angiography (DSA) of this delivery.

FIG. 2

This figure shows the size distribution of flutamide-loaded microspheres.

FIG. 3

This figure shows the in vitro release profile of flutamide from microspheres in PBS at pH 7.2 at 37° C.

FIG. 4

This figure shows the comparative FT-IR spectra of free flutamide, empty microspheres and flutamide-loaded microspheres.

FIG. 5

This figure shows a light microscopic image of a microsphere solution.

FIG. 6

This figure shows a scanning electron micrograph (SEM) image of a microsphere.

FIG. 7

This figure shows the selective posteroanterior-view angiogram of prostatic branches and injected flutamide-loaded microspheres.

FIGS. 8A and 8B

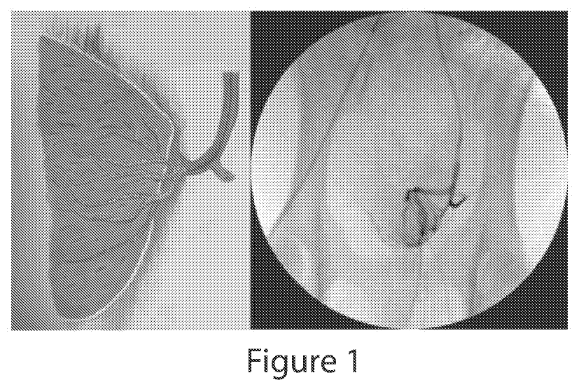

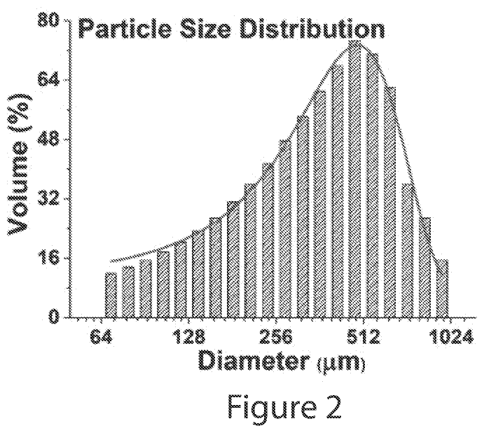

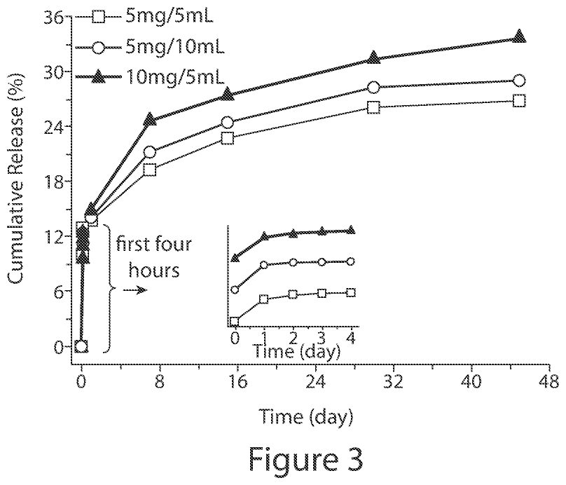

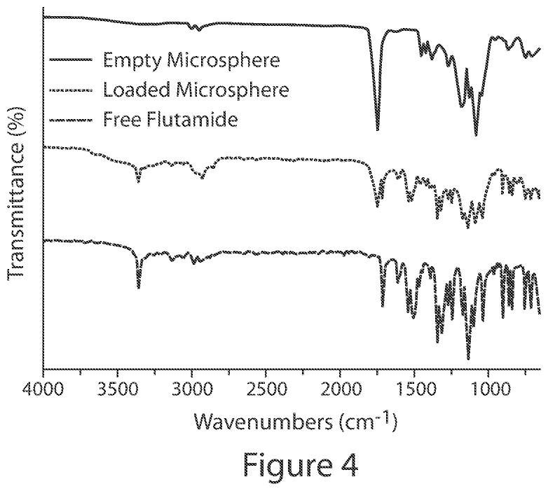

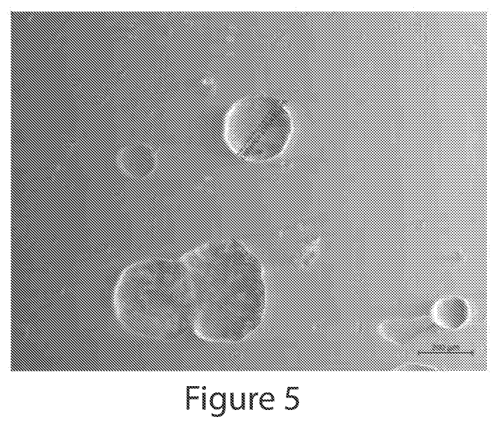

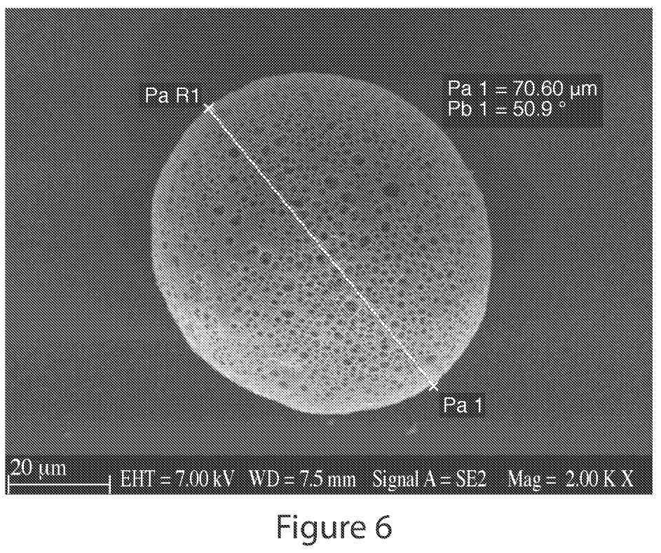

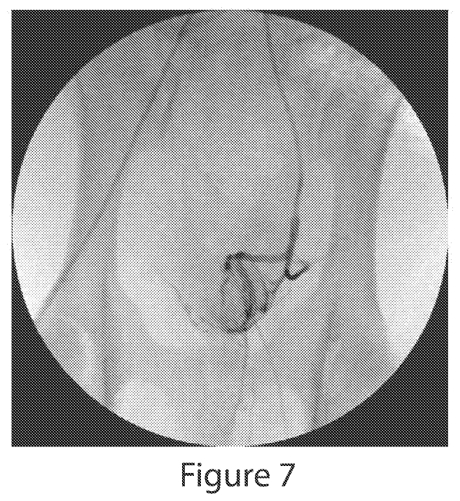

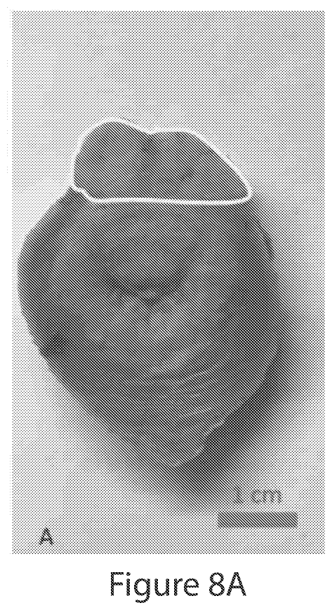

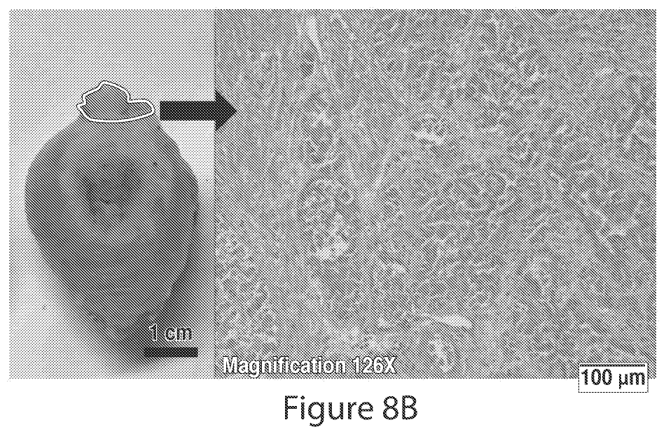

Macroscopy images of control (FIG. 8A) and treatment group (FIG. 8B) show distinctive shrinkage of the treatment group's prostate volume. The photomicrograph of a histologic section of the treatment group's prostate shows atrophy of the normal gland structure and atrophy of the residual gland tissue (via hematoxylin-eosin stain).

### DETAILED DESCRIPTION OF THE INVENTION

This invention provides new methods for treating prostate cancer and benign prostatic hyperplasia. These methods employ flutamide-carrying biodegradable microspheres to embolize patients' prostate arterial vessels.

Definitions

In this application, certain terms are used which shall have the meanings set forth as follows.

As used herein, the term “arterial vessel” means an artery, an arteriole or a capillary.

As used herein, the term “biodegradable microsphere” means a polymeric sphere that (i) has a diameter from 40 μm to 500 μm, (ii) can non-covalently carry a therapeutic agent (e.g., flutamide), and (iii) depending on its polymeric composition, degrades over a period lasting from days to months when placed in the human circulatory system. A biodegradable microsphere can be, for example, a sphere comprising PLA and PLGA (e.g., having a PLA:PLGA molar ratio of 45:55) that (i) has a diameter of 100 μm, (ii) can non-covalently contain flutamide, and (iii) degrades over a period lasting two months when embolizing a prostate arterial vessel. Biodegradable PLA/PLGA microspheres are commercially available from, among other sources, Millipore-Sigma in the form of Degradex® products (Burlington, Mass.).

As used herein, a biodegradable microsphere comprising PLA “and/or” PLGA can be a biodegradable microsphere made solely of FLA, made solely of PLGA, or made of a combination of FLA and PLGA: The higher a microsphere's PLA content, the slower it degrades and, thus, the more stable it is. Conversely, the higher a microsphere's PLGA content, the faster it degrades and the less stable it is. In one embodiment, the biodegradable microsphere is made of a combination of PLA and PLGA wherein the molar ratio of PLA to PLGA is 5:95, 10:90, 15:85, 20:80, 25:75, 30:70, 35:65, 40:60, 45:55, 50:50, 55:45, 60:40, 65:35, 70:30, 75:25, 80:20, 85:15, 90:10, or 95:5. In another embodiment, the biodegradable microsphere is made of a combination of PLA and PLGA wherein the molar ratio of PLA to PLGA is from 5:95 to 20:80, from 20:80 to 40:60, from 40:60 to 50:50, from 40:60 to 60:40, from 50:50 to 60:40, from 60:40 to 80:20, or from 80:20 to 95:5. The population of biodegradable microspheres used in this invention can be homogeneous or heterogeneous with respect to the microspheres' molar ratio of PLA to PLGA. In one embodiment, the population of biodegradable microspheres is homogeneous with respect to the microspheres' molar ratio of PLA to PLGA (e.g., the population includes only microspheres wherein the molar ratio of PLA to PLGA is 45:55). In another embodiment, the population of biodegradable microspheres is heterogeneous (e.g., the population includes both (i) microspheres wherein the molar ratio of PLA to PLGA is 45:55, and (ii) microspheres wherein the molar ratio of PLA to PLGA is 50:50). Methods of preparing biodegradable microspheres using PLA and PLGA are known, as are methods of preparing homogeneous and heterogeneous populations thereof having defined molar ratios of PLA to PLGA. Such calibrated biodegradable microspheres comprising PLA and PLGA are commercially available from, among other sources, Millipore-Sigma in the form of Degradex® products (Burlington, Mass.).

As used herein, the term “carry”, with respect to pharmaceutical flutamide and a biodegradable microsphere, means that the pharmaceutical flutamide is non-covalently bound to, or otherwise contained in or on, the biodegradable microsphere in a manner permitting release from the microsphere during its biodegradation.

As used herein, “treating” a subject afflicted with a disorder shall include, without limitation, (i) slowing, stopping or reversing the disorders progression, (ii) slowing, stopping or reversing the progression of the disorder's symptoms, (iii) reducing the likelihood of the disorder's recurrence, and/or (iv) reducing the likelihood that the disorder's symptoms will recur. In the preferred embodiment, treating a subject afflicted with a disorder means (i) reversing the disorder's progression, ideally to the point of eliminating the disorder, and/or (ii) reversing the progression of the disorder's symptoms, ideally to the point of eliminating the symptoms, and/or (iii) reducing or eliminating the likelihood of relapse. In one embodiment, successfully treating pancreatic cancer or benign prostatic hyperplasia in a human subject returns the subject's prostate to a size deemed normal for the subject's age and other relevant physical features. In another embodiment, if the treatment does not return the subject's prostate to a size deemed normal for the subject's age and other relevant physical features, successfully treating pancreatic cancer or benign prostatic hyperplasia in a human subject reduces the size of the subject's prostate by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, or at least 80%. Preferably, reduction of the size of the subject's prostate as exemplified above is measured three months after treatment.

As used herein, the term “d₉₀ value”, with respect to a population of biodegradable microspheres having a specified size range, means that 90% of the biodegradable microspheres have a diameter in the specified range.

As used herein, the term “embolize”, with respect to an arterial vessel, means to occlude blood flow through the vessel by remaining in place within it. In one embodiment of this invention, embolize means to occlude blood flow through the vessel by at least 70%, at least 75%, at least 80%, at least 85%, at least 90%, or at least 95%.

As used herein, “introducing”, with respect to biodegradable microspheres, means delivering to a specified part of the body, such as a prostate artery. Methods of introducing biodegradable microspheres to an artery to embolize that artery or arterial vessels supplied by it are known. See, e.g., Pisco, et al. (2011), Gao, et al. (2013) and Mordasini, et al. (2018).

As used herein, the term “flutamide” shall mean 4′-nitro-3′-trifluoromethyl-isobutyranilide (IUPAC name). Flutamide is sold under the brand name Eulexin®, among others, and is a non-steroidal, orally active anti-androgen. It is used to treat prostate cancer.

As used herein, the term “pharmaceutical flutamide” includes, without limitation, flutamide and pharmaceutical salts and esters thereof. Optionally, in this invention, the subject microspheres (i.e., containing pharmaceutical flutamide) can be formulated using one or more routinely used pharmaceutically acceptable carriers. Such carriers are well known to those skilled in the art.

As used herein, the term “subject” includes, without limitation, a mammal such as a human, a non-human primate, a dog, a cat, a horse, a sheep, a goat, a cow, a rabbit, a pig, a rat and a mouse. Preferably, the subject is human.

In one embodiment, the human subject is afflicted with prostate cancer, and the prostate cancer is at Stage I, Stage IIA, Stage IB, Stage IIC, Stage IIIA, Stage IIIB, Stage IIIC, Stage IVA or Stage IVB. In another embodiment, the human subject is afflicted with benign prostatic hyperplasia, and the benign prostatic hyperplasia is at Grade I, Grade II, Grade III or Grade IV. In a further embodiment, the human subject is afflicted with benign prostatic hyperplasia, and the subject's prostate has a volume of from 25 ml to 30 ml, from 30 ml to 35 ml, from 35 ml to 40 ml, from 40 ml to 45 ml, from 45 ml to 50 ml, from 50 ml to 55 ml, from 55 ml to 60 ml, from 60 ml to 65 ml, from 65 ml to 70 ml, from 70 ml to 75 ml, from 75 ml to 80 ml, from 80 ml to 85 ml, from 85 ml to 90 ml, from 90 ml to 95 ml, from 95 ml to 100 ml, or greater than 100 ml. In the preferred embodiment, the subject is at least 40 years old. In further embodiments, the subject is at least 50, at least 60, at least 70 or at least 80 years old.

As used herein, the term “therapeutically effective amount”, with respect to pharmaceutical flutamide and biodegradable microspheres, refers to the amount of pharmaceutical flutamide collectively carried by the total dose of biodegradable microspheres introduced into the subject's one or more prostate arteries. In one embodiment, the effective amount is 500 mg, 550 mg, 600 mg, 650 mg, 700 mg, 750 mg, 800 mg, 850 mg, 900 mg, 950 mg, 1,000 mg, 1,050 mg, 1,100 mg, 1,150 mg, 1,200 mg, 1,250 mg, 1,300 mg, 1,350 mg, 1,400 mg, 1,450 mg, 1,500 mg, 1,550 mg, 1,600 mg, 1,650 mg, 1,700 mg, 1,750 mg, 1,800 mg, 1,850 mg, 1,900 mg, 1,950 mg, or 2,000 mg. In another embodiment, the effective amount is from 500 mg to 550 mg, from 550 mg to 600 mg, from 600 mg to 650 mg, from 650 mg to 700 mg, from 700 mg to 750 mg, from 750 mg to 800 mg, from 800 mg to 850 mg, from 850 mg to 900 mg, from 900 mg to 950 mg, from 950 mg to 1,000 mg, from 1,000 mg to 1,050 mg, from 1,050 mg to 1,100 mg, from 1,100 mg to 1,150 mg, from 1,150 mg to 1,200 mg, from 1,200 mg to 1,250 mg, from 1,250 mg to 1,300 mg, from 1,300 mg to 1,350 mg, from 1,350 mg to 1,400 mg, from 1,400 mg to 1,450 mg, from 1,450 mg to 1,500 mg, from 1,500 mg to 1,550 mg, from 1,550 mg to 1,600 mg, from 1,600 mg to 1,650 mg, from 1,650 mg to 1,700 mg, from 1,700 mg to 1,750 mg, from 1,750 mg to 1,800 mg, from 1,800 mg to 1,850 mg, from 1,850 mg to 1,900 mg, from 1,900 mg to 1,950 mg, or from 950 mg to 2,000 mg. In a further embodiment, the effective amount is from 500 mg to 600 mg, from 600 mg to 700 mg, from 700 mg to 800 mg, from 800 mg to 900 mg, from 900 mg to 1,000 mg, from 1,000 mg to 1,100 mg, from 1,100 mg to 1,200 mg, from 1,200 mg to 1,300 mg, from 1,300 mg to 1,400 mg, from 1,400 mg to 1,500 mg, from 1,500 mg to 1,600 mg, from 1,600 mg to 1,700 mg, from 1,700 mg to 1,800 mg, from 1,800 mg to 1,900 mg, or from 1,900 mg to 2,000 mg. In a further embodiment, the effective amount is from 500 mg to 750 mg, from 750 mg to 1,000 mg, from 1,000 mg to 1,250 mg, from 1,250 mg to 1,500 mg, from 1,500 mg to 1,750 mg, or from 1,750 mg to 2,000 mg. In yet a further embodiment, the effective amount is from 500 mg to 1,000 mg, from 1,000 mg to 1,500 mg, or from 1,500 mg to 2,000 mg.

### Embodiments of the Invention

This invention solves an unmet need in the art by providing an unexpectedly superior way to treat prostate cancer and benign prostatic hyperplasia. The invention does this via embolizing the subject's prostate arterial vessels with flutamide-carrying microspheres that release flutamide over time.

Specifically, this invention provides a method for treating a prostate disorder in a subject comprising introducing biodegradable microspheres into one or more of the subject's prostate arteries, wherein the microspheres (i) have a d₉₀ value from 40 μm to 500 μm; (ii) comprise polylactic acid (PLA) and/or polylactic co-glycolic acid (PLGA); (iii) carry a therapeutically effective amount of pharmaceutical flutamide; (iv) embolize prostate arterial vessels supplied by the one or more arteries into which they are introduced; and (v) release flutamide during embolization. Preferably, the subject is human.

In the instant method, the biodegradable microspheres can be administered via one or more suitable prostate arteries. Preferably, the subject method comprises introducing the biodegradable microspheres into the subject's prostatic artery. In most male subjects, 41.5%-74.3% of the prostate gland is supplied by a common prostate/vesical arterial trunk (also named the inferior vesical artery) giving rise to the inferior vesical artery and the prostatic artery. Methods of arterial microsphere delivery generally are known, as are methods of prostate arterial microsphere delivery.

In one embodiment of the instant method, the human subject is afflicted with prostate cancer. In another embodiment, the human subject is afflicted with benign prostatic hyperplasia.

In one embodiment of the instant method, each biodegradable microsphere comprises PLA and PLGA. The PLA:PLGA molar ratio of these microspheres can be set at whatever ratio is desirable regarding stability and duration period of drug delivery. In one embodiment, each microsphere comprises PLA and PLGA at a PLA:PLGA molar ratio from 40:60 to 50:50. Preferably, the PLA:PLGA molar ratio is 45:55.

The biodegradable microspheres of the instant method can have any d₉₀ value permitting prostate embolization. Such d₉₀ values include, without limitation, 50 μm, 100 μm, 150 μm, 200 μm, 250 μm and 300 μm. In one embodiment of the instant method, the biodegradable microspheres have a d₉₀ value from 50 μm to 100 μm, from 100 μm to 150 μm, from 150 μm to 200 μm, from 200 μm to 250 μm, or from 250 μm to 300 μm. In another embodiment, the biodegradable microspheres have a d₉₀ value from 50 μm to 200 μm. In a further embodiment, the biodegradable microspheres have a d₉₀ value of 70 μm, 80 μm, 90 μm, 110 μm, 120 μm, 130 μm or 140 μm. Preferably, the biodegradable microspheres have a d₉₀ value from 70 μm to 150 μm.

In one embodiment, the therapeutically effective amount of pharmaceutical flutamide is from 500 mg to 2,000 mg.

The instant method employs biodegradable microsphere-induced embolization to permit the extended and localized release of flutamide. Ideally, the duration of embolization lasts one or more weeks (e.g., one week, two weeks, three weeks, four weeks, five weeks, six weeks, seven weeks, eight weeks, nine weeks, 10 weeks, 11 weeks or 12 weeks). Preferably, the embolization lasts from four to eight weeks. The duration of embolization can be adjusted by adjusting the PLA:PLGA molar ratios of the biodegradable microspheres. In one embodiment, the instant method is performed only once for a given subject. In another embodiment, the instant method is performed a plurality of times (e.g., two times, three times, four times, five times or more). In that embodiment, each subsequent time the method is performed, it is performed after a suitable period has lapsed since the preceding time the method was performed. This suitable time can be, for example, one month, two months, three months, four months, five months, six months, one year, or longer.

This invention also provides a method for treating prostate cancer in a human subject comprising introducing biodegradable microspheres into the subject's prostatic artery, wherein the microspheres (i) have a d₉₀ value from 70 μm to 150 μm; (ii) comprise PLA and PLGA at a PLA:PLGA molar ratio of 45:55; (iii) carry from 500 mg to 2,000 mg of pharmaceutical flutamide; (iv) embolize prostate arterial vessels supplied by the prostatic artery; and (v) release flutamide during embolization. Where applicable, the embodiments described above for the first instant method are also envisioned for this method.

This invention further provides a method for treating benign prostatic hyperplasia in a human subject comprising introducing biodegradable microspheres into the subject's prostatic artery, wherein the microspheres (i) have a d₉₀ value from 70 μm to 150 μm; (ii) comprise PLA and PLGA at a PLA:PLGA molar ratio of 45:55; (iii) carry from 500 mg to 2,000 mg of pharmaceutical flutamide; (iv) embolize prostate arterial vessels supplied by the prostatic artery; and (v) release flutamide during embolization. Where applicable, the embodiments described above for the instant methods are also envisioned for this method.

This invention provides a biodegradable microsphere, wherein the microsphere (I) has a diameter of from 40 μm to 500 μm; (ii) comprises polylactic acid (FLA) and/or polylactic co-glycolic acid (PLGA); (iii) carries pharmaceutical flutamide; (iv) embolizes a prostate arterial vessel when introduced into an artery that supplies it; and (v) releases flutamide during embolization. In the preferred embodiment, the microsphere (i) has a diameter of from 70 μm to 150 μm; and (ii) comprises PLA and PLGA at a PLA:PLGA molar ratio of 45:55. Where applicable, the embodiments described above for the instant methods are also envisioned for this microsphere.

This invention also provides a plurality of biodegradable microspheres, wherein the microspheres (i) have a d₉₀ value from 40 μm to 500 μm; (ii) comprise polylactic acid (PLA) and/or polylactic co-glycolic acid (PLGA); (iii) carry a therapeutically effective amount of pharmaceutical flutamide; (iv) embolize prostate arterial vessels when introduced into one or more arteries that supply them; and (v) release flutamide during embolization. In the preferred embodiment, the microspheres (i) have a d₉₀ value from 70 μm to 150 μm; (ii) comprise PLA and PLGA at a PLA:PLGA molar ratio of 45:55; and (iii) carry from 500 mg to 2,000 mg of pharmaceutical flutamide. Where applicable, the embodiments described above for the instant methods are also envisioned for these microspheres.

This invention provides an article of manufacture for use in treating prostate cancer by introducing biodegradable microspheres into one or more of the subject's prostate arteries. The article comprises (a) biodegradable microspheres, wherein the microspheres (i) have a d₉₀ value from 40 μm to 500 μm, (ii) comprise polylactic acid (PLA) and/or polylactic co-glycolic acid (PLGA), (iii) carry a therapeutically effective amount of pharmaceutical flutamide, (iv) embolize prostate arterial vessels supplied by the one or more prostate arteries into which they are introduced, and (v) release flutamide during embolization; and (b) a label instructing the user to introduce the biodegradable microspheres into one or more of a human subject's prostate arteries so as to treat prostate cancer in the subject. Where applicable, the embodiments described above for the instant methods are also envisioned for this article of manufacture.

This invention also provides an article of manufacture for use in treating prostate cancer in a human subject by introducing biodegradable microspheres into the subject's prostatic artery. The article comprises (a) biodegradable microspheres, wherein the microspheres (i) have a d₉₀ value from 70 μm to 150 μm, (ii) comprise PLA and PLGA at a PLA:PLGA molar ratio of 45:55, (iii) carry from 500 mg to 2,000 mg of pharmaceutical flutamide, (iv) embolize prostate arterial vessels supplied by the prostatic artery, and (v) release flutamide during embolization; and (b) a label instructing the user to introduce the biodegradable microspheres into a human subject's prostatic artery so as to treat prostate cancer in the subject. Where applicable, the embodiments described above for the instant methods are also envisioned for this article of manufacture.

This invention further provides an article of manufacture for use in treating benign prostatic hyperplasia by introducing biodegradable microspheres into one or more of the subject's prostate arteries. The article comprises (a) biodegradable microspheres, wherein the microspheres (i) have a d₉₀ value from 40 μm to 500 μm, (ii) comprise polylactic acid (PLA) and/or polylactic co-glycolic acid (PLGA), (iii) carry a therapeutically effective amount of pharmaceutical flutamide, (iv) embolize prostate arterial vessels supplied by the one or more prostate arteries into which they are introduced, and (v) release flutamide during embolization; and (b) a label instructing the user to introduce the biodegradable microspheres into one or more of a human subject's prostate arteries so as to treat benign prostatic hyperplasia in the subject. Where applicable, the embodiments described above for the instant methods are also envisioned for this article of manufacture.

This invention still further provides an article of manufacture for use in treating benign prostatic hyperplasia in a human subject by introducing biodegradable microspheres into the subject's prostatic artery. The article comprises (a) biodegradable microspheres, wherein the microspheres (i) have a d₉₀ value from 70 μm to 150 μm, (ii) comprise PLA and PLGA at a PLA:PLGA molar ratio of 45:55, (iii) carry from 500 mg to 2,000 mg of pharmaceutical flutamide, (iv) embolize prostate arterial vessels supplied by the prostatic artery, and (v) release flutamide during embolization; and (b) a label instructing the user to introduce the biodegradable microspheres into a human subject's prostatic artery so as to treat benign prostatic hyperplasia in the subject. Where applicable, the embodiments described above for the instant methods are also envisioned for this article of manufacture.

In one embodiment of the subject methods and articles of manufacture, this invention is practiced in combination with the administration of one or more therapeutic or imaging agents for treating/imaging prostate cancer or BPH. Examples of this combination approach include, without limitation, (i) practicing the subject method in combination with orally administering flutamide or a second agent; (ii) practicing the subject method in combination with intravenously administering a second agent; and (iii) practicing the subject method wherein the microspheres further comprise a second agent. In this last embodiment (iii), the second agent, for example, can be in the same microspheres as flutamide (in conjugated or unconjugated form) or in different microspheres than flutamide. The doses of oral flutamide and second agents can be, for example, the doses recommended for their individual administration or the doses set forth above for flutamide in the subject invention, as appropriate. Examples of second agents include the following, without limitation: (i) LHRH agonists (Leuprolide (Lupron, Eligard), Goserelin (Zoladex), Triptorelin (Trelstar), and Histrelin (Vantas)); (ii) LHRH antagonists (Degarelix (Firmagon), Abiraterone (Zytiga), and CYP17 inhibitor); (iii) anti-androgens (Bicalutamide (Casodex), Nilutamide (Nilandron), Enzalutamide (Xtandi), Apalutamide (Erleada), Estrogens (female hormones), and Ketoconazole (Nizoral)); (iv) chemotherapeutics (Docetaxel (Taxotere), Cabazitaxel (Jevtana), Mitoxantrone (Novantrone), and Estramustine (Emcyt)); (v) radiopharmaceutical PMSA-targeted therapeutics (¹⁷⁷Lutetium (¹⁷⁷Lu) prostate-specific membrane antigen (PSMA)-specific agents, and ²²⁵Ac prostate-specific membrane antigen (PSMA)-specific agents); and (vi) imaging agents (PET agents (F18, ZR89, and Gallium 68), optical agents (Indocyanine Green), and MRI agents (ultra-small iron particles and gadolinium)).

Wherever applicable, the methods of the subject invention may also be performed using flutamide analogs. Flutamide analogs include, without limitation, cyano (CYA) and amino analogs; 2-hydroxyflutamide; 4-nitro-3-(trifluoromethyl)-aniline; 4-nitro-6-hydroxy-3-(trifluoromethyl)-aniline; 2-methyl-N-(4′-amino-3′-[trifluoromethyl]phenyl)propanamide; 2-hydroxyflutamide; 4-nitro-3-(trifluoromethyl)phenylamine; and 2-amino-5-nitro-4-(trifluoromethyl)phenol. The dose of each flutamide analog can be, for example, the dose recommended for its individual administration or the dose set forth above for flutamide in the subject invention, as appropriate. Therefore, the various embodiments of the invention relating to flutamide-based methods also apply, mutatis mutandis, to these agents.

This invention will be better understood by reference to the examples which follow, but those skilled in the art will readily appreciate that the specific examples detailed are only illustrative of the invention as described more fully in the claims which follow thereafter.

### EXAMPLES

### Example 1—Porcine Study

Twelve large white male pigs were randomly assigned to the embolization group (n-6) or control group (n-6). Selective angiography was performed in all animals after general anesthesia was induced. In the embolization group, flutamide-loaded PLGA/PLA microspheres 150-500 microns in diameter were used to embolize the prostatic branches. One and half months later, the animals' sexual function while breeding with female pigs was subjectively evaluated. Angiographies of the embolization group prior to necropsy showed total revascularization of prostatic branches. At necropsy, the prostates were removed for size measurement and histopathologic examination. Transarterial delivery via prostatic arteries of flutamide-loaded PLGA/PLA microspheres was technically successful in all animals, without associated complications. The mean prostate volume after embolization with flutamide-loaded microspheres was reduced by 70% compared with the mean prostate volume for the group control. No significant difference in sexual function was noted between the experiment and control group. Histologic examination revealed that the PLGA/PLA microspheres had occluded the arterioles of the prostate, with disappearance of the nearby partially normal gland structure and atrophy of the residual gland tissue secondary to flutamide effect and embolization. Table 1 shows prostate volumes for pigs in embolization and control groups. Prostate volumes are in ml. Paired Student t test revealed significant difference.

TABLE 1

| Pig N. | Embolization Group (n = 6) | Control Group (n = 6) |
|---|---|---|
| 1 | 2.3 | 11.0 |
| 2 | 3.3 | 9.0 |
| 3 | 4.2 | 8.5 |
| 4 | 1.5 | 10.2 |
| 5 | 2 | 11.1 |
| 6 | 3.4 | 9.3 |
| Mean | (2.7 +/− 0.92) | (9.85 +/− 1.0) |

### Example 2—Human Study

Summary

The purpose of this project is to evaluate the safety, efficacy and feasibility of performing prostatic artery embolization using flutamide-loaded PLGA/PLA microparticles and endovascular techniques in men suffering from lower urinary tract symptoms (LUTS) due to benign prostatic hyperplasia (BPH). This study is titled “Prostate Transarterial Embolization with Flutamide Loaded PLGA/PLA microspheres Embolization for Benign Prostatic Hyperplasia.”

Study Design

Flutamide-loaded PLGA/PLA microspheres are spherical particles consisting of a PLGA and PLA polymers that are loaded with flutamide. They will be used during the study procedure, namely, prostatic artery embolization to reduce or eliminate blood flow to the prostate and provide local sustained release of flutamide.

The microspheres will be introduced via minimally invasive intra-arterial administration into prostate arteries under fluoroscopy and cone-beam CT imaging guidance.

Outcome Measures

The primary outcome measures are as follows: (1) Clinical improvement in lower urinary tract symptoms (LUTS) [time frame: 12 months], QMax (peak urinary flow rate) assessment. (2) Presence or absence of ischemic complications in the bladder, rectum, or other pelvic structures [time frame: 2 weeks]. (3) Clinical improvement in LUTS [time frame: 24 months], QMax assessment. (4) Presence or absence of ischemic complications in the bladder, rectum, or other pelvic structures [time frame: 24 months]. (5) Clinical improvement in LUTS [time frame: 12 months], IPSS (International Prostate Symptom Score). (6) Clinical improvement in LUTS [time frame: 24 months], IPSS (International Prostate Symptom Score). (7) Clinical improvement in LUTS [time frame: 12 months], QoL (quality of life question). (8) Clinical improvement in LUTS [time frame: 24 months], QoL (quality of life question). (9) Clinical improvement in LUTS [time frame: 24 months], QMax (peak urinary flow rate).

The secondary outcome measures are as follows: (1) Urine flow rate as measured by QMax [time frame: 1 month]. (2) Urine flow rate as measured by QMax [time frame: 24 months]. (3) Post-void residual measured in ml/cc [time frame: 1 month]. (4) Post-void residual measured in ml/cc [time frame: 24 months]. (5) UCLA-PCI-SF (University of California, Los Angeles Prostate Cancer Index Short Form) score [time frame: 1 month]. (6) UCLA-PCI-SF score [time frame: 24 months].

Eligibility Criteria

Male subjects must be 45-80 years old. Healthy volunteers are not accepted.

Inclusion criteria are as follows: (1) Diagnosis of LUTS from BPH for a minimum of 6 months. (2) IPSS score at initial evaluation should be greater than 7, and uroflowmetry (Qmax) of <12 mL/s (milliliters per second). (3) All prostate volumes will be >50 gm and <400 gm.

Exclusion criteria are as follows: (1) Patients with urinary tract infections (>2/year), prostatitis, or interstitial cystitis. (2) Cases of biopsy-proven prostate cancer or urethral cancer. (3) Patients on long-term narcotic analgesia, androgen therapy, or GNRH (gonadotropin-releasing hormone) analogue therapy within 2 months of study. (4) Patients who are classified as New York Heart Association Class III (Moderate), or higher. (5) Patients with history of prior pelvic irradiation. (6) Hypersensitivity reactions to contrast material not manageable with prophylaxis. (7) Patients with serum creatinine values >1.7mg/dl or glomerular filtration rates less than 50.

### REFERENCES

1. Pisco, João M., et al. “Prostatic arterial embolization to treat benign prostatic hyperplasia.” Journal of Vascular and Interventional Radiology 22.1 (2011): 11-19.

2. Gao, Yuan-an, et al. “Benign prostatic hyperplasia: prostatic arterial embolization versus transurethral resection of the prostate—a prospective, randomized, and controlled clinical trial.” Radiology 270.3 (2013): 920-928.

3. Mordasini, Livio, et al. “Prostatic Artery Embolization in the Treatment of Localized Prostate Cancer: A Bicentric Prospective Proof-of-Concept Study of 12 Patients.” Journal of Vascular and Interventional Radiology 29.5 (2018): 589-597.

4. Pisco, João, et al, “Prostate embolization as an alternative to open surgery in patients with large prostate and moderate to severe lower urinary tract symptoms.” Journal of Vascular and Interventional Radiology 27.5 (2016): 700-708.

5. Young, Shamar, and Jafar Golzarian. “Prostate arterial embolization is a viable option for treating symptoms of benign prostatic hyperplasia: Pro.” The Journal of Urology 198.1 (2017): 9-11.

What is claimed is:

1. A method for treating prostate cancer in a subject comprising introducing biodegradable microspheres into one or more of the subject's prostate arteries, wherein the microspheres (i) have a d₉₀ value from 40 μm to 500 μm; (ii) comprise polylactic acid (PLA) and/or polylactic co-glycolic acid (PLGA); (iii) carry a therapeutically effective amount of pharmaceutical flutamide; (iv) embolize prostate arterial vessels supplied by the one or more arteries into which they are introduced; and (v) release flutamide during embolization.

2. The method of claim 1, wherein the subject is human.

3. The method of claim 2, wherein the method comprises introducing the biodegradable microspheres into the subject's prostatic artery.

4. The method of claim 2, wherein each microsphere comprises PLA and PLGA.

5. The method of claim 4, wherein each microsphere comprises PLA and PLGA at a PLA:PLGA molar ratio from 40:60 to 50:50.

6. The method of claim 5, wherein each microsphere comprises PLA and PLGA at a PLA:PLGA molar ratio of 45:55.

7. The method of claim 2, wherein the biodegradable microspheres have a d₉₀ value from 50 μm to 200 μm.

8. The method of claim 7, wherein the biodegradable microspheres have a d₉₀ value from 70 μm to 150 μm.

9. The method of claim 2, wherein the therapeutically effective amount of pharmaceutical flutamide is from 500 mg to 2,000 mg.

10. The method of claim 2, wherein the embolization lasts from four to eight weeks.

11. The method of claim 1, wherein the method comprises introducing biodegradable microspheres into the subjects prostatic artery, wherein the microspheres (i) have a d₉₀ value from 70 μm to 150 μm; (ii) comprise PLA and PLGA at a PLA:PLGA molar ratio of 45:55; (iii) carry from 500 mg to 2, 000 mg of pharmaceutical flutamide; (iv) embolize prostate arterial vessels supplied by the prostatic artery; and (v) release flutamide during embolization.

12. A method for treating benign prostatic hyperplasia in a subject comprising introducing biodegradable microspheres into one or more of the subject's prostate arteries, wherein the microspheres (i) have a d₉₀ value from 40 μm to 500 μm; (ii) comprise polylactic acid (PLA) and/or polylactic co-glycolic acid (PLGA); (iii) carry a therapeutically effective amount of pharmaceutical flutamide; (iv) embolize prostate arterial vessels supplied by the one or more arteries into which they are introduced; and (v) release flutamide during embolization.

13. The method of claim 12, wherein the subject is human.

14. The method of claim 13, wherein the method comprises introducing the biodegradable microspheres into the subject's prostatic artery.

15. The method of claim 13, wherein each microsphere comprises PLA and PLGA.

16. The method of claim 15, wherein each microsphere comprises PLA and PLGA at a PLA:PLGA molar ratio from 40:60 to 50:50.

17. The method of claim 16, wherein each microsphere comprises PLA and PLGA at a PLA:PLGA molar ratio of 45:55.

18. The method of claim 13, wherein the biodegradable microspheres have a d₉₀ value from 50 μm to 200 μm.

19. The method of claim 18, wherein the biodegradable microspheres have a d₉₀ value from 70 μm to 150 μm.

20. The method of claim 13, wherein the therapeutically effective amount of pharmaceutical flutamide is from 500 mg to 2,000 mg.

21. The method of claim 13, wherein the embolization lasts from four to eight weeks.

22. The method of claim 12, wherein the method comprises introducing biodegradable microspheres into the subject's prostatic artery, wherein the microspheres (i) have a d₉₀ value from 70 μm to 150 μm; (ii) comprise PLA and PLGA at a PLA:PLGA molar ratio of 45:55; (iii) carry from 500 mg to 2,000 mg of pharmaceutical flutamide; (iv) embolize prostate arterial vessels supplied by the prostatic artery; and (v) release flutamide during embolization.

23. A biodegradable microsphere, wherein the microsphere (i) has a diameter of from 40 μm to 500 μm; (ii) comprises polylactic acid (FLA) and/or polylactic co-glycolic acid (PLGA); (iii) carries pharmaceutical flutamide; (iv) embolizes a prostate arterial vessel when introduced into an artery that supplies it; and (v) releases flutamide during embolization.

24. The biodegradable microsphere of claim 23, wherein the microsphere (i) has a diameter of from 70 μm to 150 μm; and (ii) comprises PLA and PLGA at a PLA:PLGA molar ratio of 45:55.

25. A plurality of biodegradable microspheres, wherein the microspheres (i) have a d₉₀ value from 40 μm to 500 μm; (ii) comprise polylactic acid (PLA) and/or polylactic co-glycolic acid (PLGA); (iii) carry a therapeutically effective amount of pharmaceutical flutamide; (iv) embolize prostate arterial vessels when introduced into one or more arteries that supply them; and (v) release flutamide during embolization.

26. The plurality of biodegradable microspheres of claim 25, wherein the microspheres (i) have a d₉₀ value from 70 μm to 150 μm; (ii) comprise PLA and PLGA at a PLA:PLGA molar ratio of 45:55; and (iii) carry from 500 mg to 2,000 mg of pharmaceutical flutamide.
